(19) United States
(12) Patent Application Publication
(10) Pub. No.: US 2004/0115616 A1
(43) Pub. Date: Jun. 17, 2004

(54) Stem-loop vector system

(76) Inventor: Timothy Albert Holton, Queensland (AU)

Correspondence Address: Michael R Ward, Morrison & Foerster, 425 Market Street, San Francisco, CA, 94105-2482, US

(21) Appl. No.: 10/333,144

(22) PCT Filed: Sep. 27, 2002
(86) PCT No.: PCT/AU02/01326

(30) Foreign Application Priority Data
Sep. 27, 2001 (AU) PR 7966/01
Aug. 28, 2002 (AU) 2002951039

(51) Int. Cl. C12Q 1/70; G01N 33/53; C12N 15/86

(52) U.S. Cl. 435/5; 435/7.1; 435/456; 435/69.1

(57) ABSTRACT

The present invention relates generally to a method for generating a nucleic acid library. More particularly, the present invention provides a library of eukaryotic-derived nucleic acid molecules inserted into vectors and maintained in a prokaryotic microorganism or as isolated and/or purified nucleic acid molecules. Such molecules are useful for transforming or otherwise being introduced to eukaryotic cells which can then be screened for transcriptional or post-transcriptional gene silencing (TGS or PTGS) events.

### FIELD OF THE INVENTION

The present invention relates generally to a method for generating a nucleic acid library. More particularly, the present invention provides a library of eukaryotic-derived nucleic acid molecules inserted into vectors and maintained in a prokaryotic microorganism or as isolated and/or purified nucleic acid molecules. Such molecules are useful for transforming or otherwise being introduced to eukaryotic cells which can then be screened for transcriptional or post-transcriptional gene silencing (TGS or PTGS) events.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The increasing sophistication of recombinant DNA techniques has provided significant progress in understanding the mechanisms regulating eukaryotic gene expression. This is greatly facilitating research and development in the plant, agricultural, medical and veterinary industries. One important aspect is the development of means to alter the phenotype of a cell or group of cells by modulating the expression of genetic material. A myriad of desirable phenotypic traits is potentially obtainable following selective inactivation of gene expression. However, whilst advances have been made in regulating gene expression, much less progress has been made in the actual manipulation of gene expression to produce such novel traits. Moreover, only limited means, by which human intervention may lead to a modulation of the level of eukaryotic gene expression, have been available.

In the literature, the term “gene silencing” is frequently used. This has generally been done, however, in the absence of an appreciation of whether the gene silencing events were acting in cis or in trans. This is relevant to the commercial exploitation of gene silencing technology, since cis inactivation events are of less usefulness than events in trans. For example, there is less likelihood of success in targeting endogenous genes (e.g. plant genes) or exogenous genes (e.g. genes from pathogens) using techniques that require cis inactivation.

One approach to gene inactivation (i.e. the inactivation of gene expression) utilizes anti-sense nucleic acid molecules directed to complementary mRNA transcripts. It has been postulated that a double-stranded mRNA forms by base pairing between the complementary nucleotide sequences to produce a complex which is translated at low efficiency and/or degraded by intracellular ribonuclease enzymes prior to being translated.

In an alternative approach, the expression of an endogenous gene in a cell, tissue or organ may be suppressed when one or more copies of the gene, or one or more copies of a substantially similar gene, are introduced into the cell. The transgene is expressed as sense RNA. This appears to involve mechanistically heterogeneous processes. For example, this approach has been postulated to involve either repression at the level of transcription, in which somatically-heritable repressed states of chromatin are formed or alternatively, repression following transcription, in which case transcription initiation occurs normally but RNA products are subsequently eliminated. In other words, gene inactivation may occur in cis or in trans. For cis inactivation, only the target gene is inactivated and other similar genes dispersed throughout the genome are not affected. In contrast, inactivation in trans occurs when one or more genes dispersed throughout the genome and sharing homology with a particular target sequence are also inactivated.

The term “co-suppression” is used to describe the latter form of PTGS. Expression of such transgene sequences results in inactivation of homologous genes, i.e. a sequence specific in trans inactivation of gene expression (Napoli et al., The Plant Cell 4: 279-289, 1990; van der Krol et al., The Plant Cell 4: 291-299 1990). The molecular phenotype of cells in which this occurs is well described in plant systems and the disappearance of mRNA sequences is thought to occur as a consequence of activation of a sequence-specific RNA degradative system (Lindbo et al, The Plant Cell 5: 1749-1759, 1993; Waterhouse et al., Proc. Natl. Acad. Sci. USA 95: 13959-13964, 1999).

In essence, co-suppression is thought to involve the generation of interference RNA (RNAi). RNAi refers to PTGS induced by double-stranded RNA (dsRNA). It has been shown that injection of dsRNA into C. elegans leads to sequence-specific gene silencing (Fire et al., Nature 391: 806-811, 1998). Ingestion of dsRNA (Timmons and Fire, Nature 395: 854, 1998) or bacteria expressing gene constructs to produce dsRNA (Timmons et al., Gene 263: 103-112, 2001) also leads to PTGS. RNAi has since been demonstrated to be effective in a range of organisms including Drosophila (Caplen et al., Gene 252: 95-105, 2000; Fortler and Belote, Genesis 264: 240-244, 2000), spiders (Schoppmeier and Damen, Development Genes & Evolution 211: 76-82, 2001) and mammals (Elbashir et al., Nature 411: 494-498, 2001).

The frequency of PTGS induced by transgene expression can be increased by use of hairpin or inverted repeat (IR) gene constructs (Singh et al., Biochemcial Society Transactions 28: 925-927, 2000; Smith et al., Nature 407: 319-320, 2000). Such constructs have been shown to produce close to 100% PTGS frequencies in plants. Inverted repeat constructs are also effective in animals, for example, Drosophila (Fortier and Belote, 2000, supra) and C. elegans (Timmons et al., 2001, supra). However, creation of an IR construct can only be achieved one gene at a time and requires multiple cloning steps. Accordingly, current methods for generating IR gene constructs are time consuming and labour intensive. There are no known methods for creating a library of inverted repeat or hairpin gene constructs in a single cloning step.

U.S. Pat. No. 6,054,299 describes a method for constructing a stem-loop cloning vector. The vector is useful for producing a single-stranded nucleic acid molecule that is to be cis-activated by a desired double-stranded genetic element, for example, a promoter. The nucleic acid molecule is cloned into a double-stranded replicative form of the vector between a pair of IR sequences. The IR sequences encode the double-stranded genetic element. When expressed as a single-stranded DNA, the cloned nucleic acid is located in a single-stranded “loop” region of a “stem-loop” structure. U.S. Pat. No. 6,054,299 does not describe or allude to cloning of a DNA fragment into the double-stranded stem of the “single-stranded” form of the vector, nor does the disclosure provide a means for cloning a double-stranded nucleic acid to create an inverted repeat.

There has been considerable confusion within the animal literature regarding the term “co-suppression” (Bingham, Cell 90: 385-387, 1997). In fact, until relatively recently, “co-suppression”, as defined by the specific molecular phenotype of gene transcription without translation, was considered not to occur in mammalian systems. It had been described only in plant systems and in a lower eukaryote, Neurospera (Cogoni et al., EMBO J. 15: 3153-3163, 1996; Cogoni & Mancino, Proc. Natl. Acad. Sci. USA 94: 10233-10238, 1997). However, work over the past few years has shown that comparable post-transcriptional inactivation events do, in fact, occur in mammalian and other animal systems.

A range of different genetic constructs has been demonstrated to be efficacious in systems designed to down-regulate or otherwise modulate gene expression in either plant or animal cells.

Until now, however, use of such constructs in relation to a target endogenous sequence has relied on generating particular sequence-specific genetic molecules designed to interfere with the expression of a known target sequence, requiring that both the particular nucleic acid sequence and the biological function of a targeted endogenous gene be known. Furthermore, the manufacture of such constructs requires multiple cloning steps and is generally done on a gene-by-gene (i.e. trait-by-trait) basis, addressing only one trait/phenotype at a time and making the entire process extremely labour-intensive.

With the advent of sophisticated means of high throughput screening and micro-array technologies, very large numbers of molecules are able to be generated and screened for desired characteristics simultaneously and rapidly, thereby greatly increasing the efficiency of commercially-directed research protocols and product development. For the types of gene-modulating constructs described above to be routinely applied in diagnostically and therapeutically useful ways, there is a need for the creation of much more rapid and predictable means of generating libraries of potentially functionally relevant genetic molecules.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc.

In work leading up to the present invention, the inventor has developed a vector system useful in the generation of nucleic acid libraries comprising eukaryotic-derived genetic sequences. These sequences may comprise cDNA and/or genomic DNA. The genomic DNA may comprise one or more promoter or other regulatory or non-transcribed regions. When introduced into particular eukaryotic cells, the nucleic acid molecules in the library may generate partially double-stranded RNA transcripts. The library is useful, therefore, for producing nucleic acid molecules that result in gene silencing in eukaryotic cells. The RNA transcripts of the present invention are referred to herein as “co-suppression effectors”. They may take the form of a “hairpin-shaped construct” or a “perfect hairpin”, as described hereinafter. Silencing may occur via PTGS, where the library comprises genetic sequences derived from, for example, cDNA or genomic DNA corresponding to an amino acid-encoding or RNA-encoding region of a genetic sequence. Alternatively, it may occur via TGS, where the library comprises genetic sequences derived from, for example a non-transcribed promoter or other regulatory DNA region. In this second case, the library may generate partially double-stranded RNA transcripts targeted at, for example, a non-transcribed promoter region, resulting eventually in TGS, such as via DNA methylation.

The present invention provides, therefore, a range of genetic molecules referred to herein respectively as a co-suppression vector, co-suppression constructs (in double-stranded and partially single-stranded forms), a co-suppression library and co-suppression effectors.

The co-suppression vector may comprise a single-stranded loop portion and a single-stranded replicon portion separated by a double-stranded DNA portion comprising at least one restriction endonuclease site. This is referred to herein as a “co-suppression vector”. When maintained in a prokaryotic organism, in the absence of helper phage, the vector may be in a double-stranded form.

Into the double-stranded portion of the co-suppression vector may be introduced eukaryotic DNA. The eukaryotic DNA may comprise cDNA or genomic DNA. This then becomes the partially single-stranded form of the co-suppression construct and is referred to herein as “ss co-suppression construct” or “co-suppression construct (i)”. The resulting recombinant molecule may be introduced into, for example, a prokaryotic microorganism to produce a library of double-stranded co-suppression constructs comprising the eukaryotic DNA. This is the double-stranded form of the co-suppression construct and is referred to herein as “ds co-suppression construct” or “co-suppression construct (ii)”.

The co-suppression constructs of the invention are generated from a double-stranded DNA cloning vector, according to the method described herein.

In one particular embodiment, the present invention may be described as including the following steps:

(i) conversion of a double-stranded replicative circular DNA cloning vector, comprising an inverted repeat sequence, into a single-stranded form;

(ii) treatment of said single-stranded form such that self-complementary sequences derived from the inverted repeat (IR) sequence anneal to form a region of double-stranded nucleic acid;

(iii) cleavage of the double-stranded region formed in step (ii) by one or more restriction enzymes to form a vector stem-loop portion and a spacer stem-loop portion;

(iv) ligation of (iii) with double-stranded DNA fragments containing termini compatible with the vector and spacer stem-loops;

(v) conversion of recombinant nucleic acid molecules derived from step (iv) into double-stranded circular form, thereby generating a nucleic acid construct containing an IR of the cloned double-stranded fragment(s), referred to herein as a co-suppression construct or an IR DNA construct.

Preferably, the double-stranded region formed in step (ii) contains at least one restriction enzyme recognition site and is of sufficient length to stabilise the stein/loop structures formed by subsequent cleavage in step (iii).

Where two restriction enzymes are used in step (iii), a double-stranded linear fragment will be concomitantly released. Most restriction enzymes only cleave double-stranded DNA. Hence, cleavage in step (iii) should only occur in the annealed double-stranded region, not in other single-stranded regions of the vector, even if there are additional restriction endonuclease recognition sites in the single-stranded loop regions.

The conversion of recombinant nucleic acid molecules into double-stranded form, effected in step (v), can be achieved either in vitro or by transformation of a host cell which will convert it to double-stranded form as part of the replicative process.

The double-stranded DNA cloning vector, with which the method of the present invention begins, may comprise one or more promoters operable in eukaryotic cells. A co-suppression construct generated therefrom may, therefore, also comprise one or more promoters operable in eukaryotic cells and operably linked to a portion of the co-suppression vector upstream of the IRs.

Consequently, upon introduction of the co-suppression library into a suitable eukaryotic cell, expression mediated by a eukaryotic promoter results in double-stranded eukaryotic RNA with or without a stem loop, depending on whether or not the spacer is an intron. The double-stranded eukaryotic RNA with or without a stem loop is referred to herein as a “co-suppression effector”.

Eukaryotic cells carrying the co-suppression effector RNA are then screened for the effects of PTGS or TGS.

In an alternative embodiment, the double-stranded DNA cloning vector from which the co-suppression constructs of the invention are generated, may comprise one or more promoters operable in prokaryotic cells. In this case, a co-suppression construct generated therefrom may, therefore, also comprise one or more promoters operable in prokaryotic cells and operably linked to a portion of the co-suppression vector upstream of the IRs.

Accordingly, when eukaryotic DNA is introduced into the double-stranded portion of the partially single-stranded vector, the resulting recombinant molecule, when introduced into a prokaryotic microorganism, produces a double-stranded co-suppression library comprising the introduced eukaryotic and prokaryotic DNA. One or more promoters operable in prokaryotic cells are comprised within the introduced DNA. Expression of this form of the co-suppression library in a prokaryotic cell is then mediated by a prokaryotic promoter, again resulting in co-suppression effectors. This form of the co-suppression library may be used in feeding situations, where the ingestion of the library by a eukaryotic organism may result in the generation of co-suppression effector RNAs which interact with the nucleic acid material of the eukaryotic organism, possibly resulting in PTGS.

In either embodiment, the co-suppression effector RNAs may cause gene silencing, either via PTGS or via TGS, depending on the identity of the genetic sequences comprised in the co-suppression construct from which the co-suppression effector RNA was derived.

The present invention provides, therefore, a co-suppression library either in prokaryotic microorganisms or as nucleic acid molecules in an isolated or purified form. The co-suppression library comprises eukaryotic DNA, generally randomly generated by digestion of a particular eukaryotic genome. The co-suppression library may also further comprise prokaryotic DNA. The generation of the library does not require any prior knowledge of a target gene. All that is required is an appropriate eukaryotic indicator cell line. Such a cell line is used to identify TGS or PTGS via a detectable trait or reporter signal.

The present invention further provides isolated or purified prokaryotic cells comprising the co-suppression library of double-stranded co-suppression constructs, single-stranded co-suppression constructs or co-suppression vectors. The present invention further provides eukaryotic or prokaryotic cells comprising co-suppression effectors.

A summary of sequence identifiers and a glossary of important terms used throughout the subject specification are provided in Tables 1 and 2, respectively.

TABLE 1
Summary of sequence identifiers

| SEQUENCE ID NO: | DESCRIPTION |
|---|---|
| 1 | Acc65I |
| 2 | BsiWI |
| 3 | PpoAcc primer |
| 4 | PpoBsi primer |
| 5 | PPO-Srf-F primer |
| 6 | PPO-Srf-R primer |
| 7 | MCS-Sac-Dra |
| 8 | MCS-Kpn-Mlu |

TABLE 2
Glossary of terms

| TERM | MEANING |
|---|---|
| double-stranded | DNA vehicle comprising or into which has been |
| DNA cloning | cloned particular useful features such as, for |
| vector | example, a selectable marker gene, inverted repeat |
| | sequences, multiple cloning sites, prokaryotic |
| | and/or eukaryotic promoter regions, stop codon, |
| | inter alia |
| co-suppression | single-stranded form of the DNA cloning vector, |
| vector | having a double-stranded stem portion into which |
| | eukaryotic DNA may be inserted |
| co-suppression | a co-suppression vector comprising eukaryotic |
| construct (i) | DNA |
| co-suppression | the double-stranded form of a co-suppression |
| construct (ii) | construct (i) |
| co-suppression | a mixture of co-suppression constructs (ii), either |
| library | in a prokaryotic organism or in isolated form |
| co-suppression | an RNA molecule transcribed from co-suppression |
| effector | construct (ii) |

### BRIEF DESCRIPTION OF THE FIGURES

FIG. 1 is a diagrammatic representation of the protocol for generating a library of eukaryotic nucleic acid molecules (referred to herein as co-suppression constructs and co-suppression effectors) in prokaryotic cells and testing these in eukaryotic cells.

FIG. 2 is a diagrammatic representation showing a production of a single-stranded co-suppression construct by a single cloning step. A double-stranded DNA cloning vector (refer to FIG. 7) is converted into a predominantly single-stranded form and a self-complementary inverted repeat (IR) region is allowed to anneal to form a double-stranded region. One or more restriction enzyme recognition sites within the double-stranded region is then cut with an appropriate enzyme(s), producing two stem-and-loop structures. Ligation of a stem-and-loop with a compatible double-stranded DNA fragment produces a double-stranded region flanked by single-stranded loops.

FIG. 3 is a diagrammatic representation showing one end of a double-stranded nucleic acid fragment (e.g. heterologous nucleic acid) that is ligated to a compatible stem-and-loop nucleic acid to form a stem-and-loop DNA molecule (referred to herein as a co-suppression construct). The construct is converted to a double-stranded form by synthesis of the complementary strand thereby creating a spacer DNA region flanked by an IR of the original double-stranded DNA polynucleotide. Synthesis of the complementary strand may be achieved either in vitro using, for example, a DNA polymerase and a suitable primer or in vivo using, for example, the DNA replication mechanism provided by a host cell.

FIG. 4 is a diagrammatic representation showing conversion of the nucleic acid shown in FIG. 2 to a double-stranded form by synthesis of a complementary strand to produce a double-stranded co-suppression construct comprising a spacer region flanked by IRs of the cloned DNA fragment.

FIG. 5 is a diagrammatic representation showing a single-stranded co-suppression vector, comprising a short IR that does not require an intervening spacer region to enable replication in bacteria. This single-stranded vector can be cut with appropriate restriction enzyme(s) to produce a plasmid (replicon)+stem portion, which can subsequently be either self-ligated or ligated with a compatible DNA fragment comprising a spacer+-stem portion.

FIG. 6 is a diagrammatic representation showing production of a stem-and-loop structure via polymerase chain reaction (PCR). PCR amplification of DNA fragments with two primers comprising regions of sequence identity results in creation of amplification products comprising IRs at their termini. PCR amplification of one strand, using a single primer results in production of predominantly single-stranded products, which can self-anneal to form stem-and-loop structures. These structures can then be first digested with a restriction enzyme or directly ligated with compatible fragments for the creation of co-suppression constructs.

FIG. 7 is a diagrammatic representation showing an example of cloning steps that can be used to produce a double-stranded DNA cloning vector.

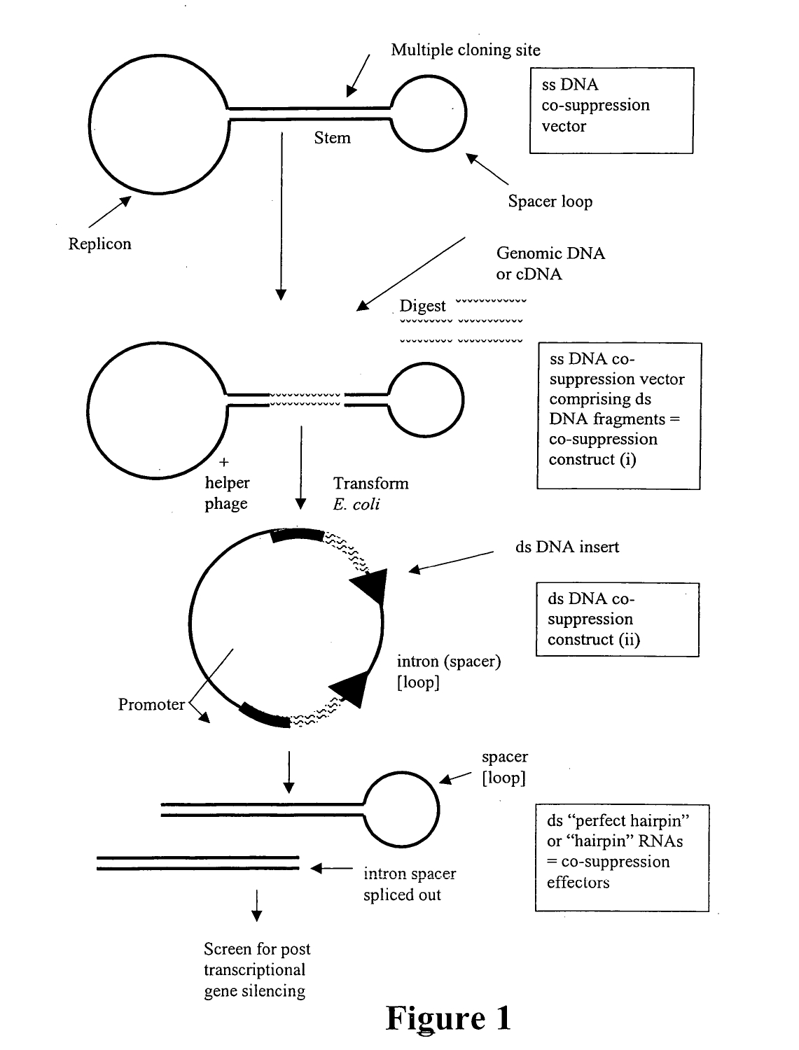

### DETAILED DESCRIPTION OF THE PREFERRED EMBODIMDENTS

The present invention is predicated in part on the development of a vector useful for generating a nucleic acid library of eukaryotic genetic sequences and/or a combination of eukaryotic and prokaryotic genetic sequences, in double-stranded DNA form. The vector is referred to herein as a “co-suppression vector”. The library of eukaryotic DNA inserts is referred to herein as a “co-suppression library”. Any individual co-suppression vector comprising a eukaryotic DNA insert is referred to herein as co-suppression construct. Such co-suppression constructs may, alternatively, be referred to as inverted repeat (IR) DNA constructs. Conveniently, the co-suppression library is maintained in a prokaryotic cell. The present invention extends, however, to the co-suppression library in isolated and/or purified form.

The co-suppression vector permits the generation of a co-suppression library of eukaryotic genetic sequences. Introduction of a particular co-suppression library to eukaryotic cells, followed by expression, results in RNA having a double-stranded portion. The ability of the co-suppression library to introduce or otherwise facilitate gene silencing of particular eukaryotic genes can then be screened for. No knowledge of the eukaryotic sequences is required. The library, in one form, comprises randomly generated representatives of a eukaryotic genome. When introduced into a particular eukaryotic cell line, PTGS or TGS is monitored by, for example, alteration of a particular trait or change in a particular signal.

Accordingly, in one embodiment, the present invention contemplates a method for generating a library of viral- or eukaryotic-derived nucleic acid molecules in a suitable cell, said method comprising the steps of:—

(i) generating a vector wherein, in vitro, the vector comprises a single-stranded replicon portion and a single-stranded loop portion separated by double-stranded stem portion comprising at least one restriction endonuclease site;

(ii) digesting said partially single-stranded vector with at least one restriction endonuclease and admixing therewith double-stranded genomic DNA or cDNA derived from a eukaryotic cell and digested with the same restriction endonuclease or other enzyme or under conditions providing compatible 3′ and 5′ end portions for ligation into the restricted partially single-stranded vector and subjecting said admixture to ligation conditions to generate the partially single-stranded vector comprising double-stranded genomic DNA or cDNA fragments inserted into the double-stranded portion of said vector; and

(iii) introducing the ligated admixture of (ii) into said suitable cell under conditions to permit the generation of a double-stranded replicative form of said partially single-stranded vector comprising double-stranded genomic DNA or cDNA fragments.

In an alternative embodiment, the double-stranded replicative form of said partially single-stranded vector comprising double-stranded genomic DNA or cDNA fragments is first generated in vitro from the ligated admixture. The replicative form is then subsequently introduced into a suitable cell.

Suitable cells comprise eukaryotic cells and prokaryotic microorganisms.

Accordingly, in another embodiment, the present invention contemplates a method for generating a library of viral- or eukaryotic-derived nucleic acid molecules in a suitable cell, said method comprising the steps of:—

(i) generating a vector wherein, in vitro, the vector comprises a single-stranded replicon portion and a single-stranded loop portion separated by double-stranded stem portion comprising at least one restriction endonuclease site;

(ii) digesting said partially single-stranded vector with the at least one restriction endonuclease and admixing therewith double-stranded genomic DNA or cDNA derived from a eukaryotic cell and digested with the same restriction endonuclease or other enzyme or under conditions providing compatible 3′ and 5′ end portions for ligation into the restricted partially single-stranded vector and subjecting said admixture to ligation conditions to generate the partially single-stranded vector comprising double-stranded genomic DNA or cDNA fragments inserted into the double-stranded portion of said vector; and

(iii) generating, in vitro, a double-stranded replicative form of said partially single-stranded vector comprising double-stranded genomic DNA or cDNA fragments from the ligated admixture of (ii), prior to introducing the resulting double-stranded replicative form into said suitable cell.

The double-stranded stem portion of step (i) arises from self-annealing of complementary sequences derived from the IR sequences introduced into a double-stranded DNA cloning vector. Generation and subsequent use of DNA cloning vectors is described hereinafter.

The partially single-stranded vector may be digested with a single restriction endonuclease or a combination of two or more restriction endonucleases (step (ii)).

The resulting double-stranded replicative form generated by step (iii) comprises exogenous eukaryotic DNA and is referred to herein as a double-stranded co-suppression construct. The population of molecules represents the co-suppression library. The co-suppression library may be isolated or purified nucleic acid molecules, or may be a culture of cells comprising same.

In one preferred embodiment, the culture comprises a prokaryotic microorganism.

Any suitable prokaryotic microorganism may be utilized as a host for the library of nucleic acid molecules generated by the above method, the requirement being that when a partially single-stranded form of the co-suppression vector is required, the microorganism has to support formation of a single-stranded replicative form, generally with the use of a helper phage. At other times, any other prokaryotic microorganism may be employed.

The generation of a partially single-stranded vector having a single-stranded replicon portion and a single-stranded spacer-loop portion separated by a double-stranded stem comprising at least one restriction endonuclease site (step (i)) is initiated by first obtaining a double-stranded DNA cloning vector having a multiple cloning site. A “multiple cloning site” means a multiplicity of two or more restriction endonuclease sites and preferably one or more unique restriction endonuclease sites which, upon digestion, results in a vector being cleaved only within the multiple cloning site.

A range of vectors may be employed at this step but a pBluescript vector is particularly useful. One particular pBluescript vector comprises the multiple cloning site comprising the restriction endonuclease sites BssHI, Acc651, ApaI, XhoI, SalI, EcoRI, NotI and SacI. The multiple cloning site is flanked by BssHI sites.

A spacer nucleic acid molecule is then cloned into the multiple cloning site. Preferably, but certainly not exclusively, the spacer molecule is or comprises an intron.

An intron is useful in that upon expression in a eukaryotic cell, the intronic spacer is spliced out from the transcript. A spacer may be regarded as a “hinge” to permit homologous nucleotide sequences on two strands separated by the hinge to fold back and anneal to each other. Preferably, the length of the spacer is such as to not adversely impact on the efficiency of self-annealing of the complementary homologous sequences.

The PPO intron from the pineapple PPO gene is particularly useful as a spacer element although any intron may be employed.

The spacer is inserted into the multiple cloning site leaving, generally but not exclusively, one or more unique restriction sites adjacent both sides of the spacer. If more than one restriction site and, hence, restriction endonuclease, is used, directional cloning of digested DNA fragments is facilitated. Moreover, a stretch of the double-stranded stem may be able to be removed, and inserts of predetermined length—such as may be generated following size fractionation—may be cloned therein.

The double-stranded cloning vector being constructed also requires homologous nucleotide sequences flanking the spacer. The multiple cloning site is regarded as the first of these nucleotide sequences. The introduction of the same or a homologous multiple cloning site, in inverse orientation, on the other side of the spacer to the one within or adjacent the first multiple cloning site, is required. The spacer is preferably inserted into one end of the multiple cloning site. To achieve this, the next step in constructing the cloning vector is to digest it with appropriate restriction endonuclease enzymes, to isolate a fragment comprising the multiple cloning site or part thereof and the spacer. The same or homologous multiple cloning site is then excised from another vector and a three-way ligation reaction initiated with a cloning vector so as to produce a cloning vector having two IRs in the form of the same or homologous multiple cloning sites flanking the spacer. For a diagrammatic representation of this procedure, refer to FIG. 7.

There are a number of different ways in which the double-stranded DNA cloning vector may be generated and the present invention is not to be limited to any one means of production.

Accordingly, another aspect of the present invention contemplates a method for generating a double-stranded DNA cloning vector useful for generating a co-suppression library, said method comprising introducing into a double-stranded vector, which is optionally capable of generating single-stranded replicative intermediates in the presence of a helper phage, two homologous nucleotide sequences flanking a spacer nucleotide sequence such that, when in single-stranded form, the spacer nucleotide sequence permits the two homologous nucleotide sequences to anneal together to create a partially double-stranded molecule.

In one embodiment, the double-stranded DNA cloning vector comprises some but not all the genetic material required to replicate via a single-stranded intermediate. Consequently, in the presence of a helper phage in a prokaryotic microorganism, a single-stranded replicative intermediate is generated. This is a co-suppression vector.

Following standard biochemical extraction and precipitation techniques, the co-suppression vector is isolated. In vitro, the single-stranded co-suppression vector may comprise a double-stranded portion comprising the two same or homologous multiple cloning sites. The “replicon portion” of the co-suppression vector is derived from the double-stranded DNA cloning vector initially employed and permits replication in a host microbial microorganism. The spacer “loop portion” comprises the nucleic acid spacer sequence which was cloned into, and hence separated, the multiple cloning site, and the “double-stranded stem portion” comprises the one or more restriction endonuclease recognition sequences from two of the same or homologous multiple cloning sites.

These restriction endonuclease recognition sequences may then serve as cloning sites, using standard cloning procedures well known in the art. Any restricted double-stranded genomic DNA or cDNA preparation may be ligated therein, provided that it has compatible 3′ and 5′ ends. Both sticky ends and ends that have been appropriately blunted may be comprised within the term “compatible”. Reference to a “cDNA” includes cDNA corresponding to a single gene as well as to two or more genes from the genome of an organism.

In an alternative embodiment, a co-suppression construct may be generated in vitro by “nicking” of one strand of a double-stranded DNA cloning vector. The nicked strand is then digested with, for example, Exonuclease III, to leave a single-stranded circular DNA remaining. Upon exposure to annealing conditions, a self-complementary complex forms, comprising two single-stranded “loop portions” joined by a double-stranded “stem”. Digestion with a suitable enzyme(s) produces a “replicon stem-loop” and a “spacer stem-loop”, which may again be ligated with compatible eukaryotic DNA.

In one preferred embodiment, restriction endonucleases having non-palindromic recognition sequences are utilised. With such enzymes it is possible to reduce the background that is sometimes observed due to, for example, ligation of replicon stem-loops to each other.

For the construction of a library of co-suppression constructs according to the methods of the present invention, a genomic or cDNA preparation from any eukaryotic organism may be fragmented. The choice of eukaryotic organism will be determined only by the species and target of interest. Suitable eukaryotic cells include inter alia those derived from plants as well as animals, such as mouse and livestock animals as well as human animals and invertebrate animals such as insects and nematodes.

Accordingly, another aspect of the present invention provides a co-suppression construct comprising two single-stranded DNA loop portions separated by a double-stranded portion wherein the double-stranded portion comprises one or more restriction endonuclease sites into which has been introduced a double-stranded DNA fragment.

This is the partially single-stranded form of the co-suppression construct, also referred to as co-suppression construct (i). In a suitable cell such as, for example, a prokaryotic microorganism, this co-suppression construct is converted into a double-stranded form, referred to as a co-suppression construct (ii) or, alternatively, an IR DNA construct. The present invention extends to a mixture of single-stranded and double-stranded forms as may exist in, for example, M13. As a result of carrying out the methods of the present invention, a library of such nucleic acid molecules may be generated.

A co-suppression construct may also be replicated in vitro, such as via rolling circle replication, to generate a concatamer comprising multiple copies of one strand of a co-suppression construct. Upon exposure to annealing conditions, a nucleic acid complex forms, comprising multiple “stem-loop” portions. Digestion with a suitable enzyme provides a source of spacer-stem portions for ligation to compatibly digested replicon-stem portion of a co-suppression construct. Thereby hybrid co-suppression constructs may be generated.

Stem-loop structures may also be generated via PCR amplification. Amplification products may be ligated to blunt-ended double-stranded DNA fragments and/or to replicon-stem portions to form a co-suppression construct. Alternatively, a single-stranded spacer-loop with self-complementary ends may be ligated to a single-stranded cDNA polynucleotide and used to prime second strand synthesis. Double-stranded stem portions thereby generated may be ligated to double-stranded DNA fragments and/or to replicon-stem portions to form a co-suppression construct.

Hence, a co-suppression construct may be generated by any number of means and may be in any number of forms. Preferably, expression of the double-stranded form results in the formation of RNA comprising either a stem-loop in the form of a hairpin, or a perfect hairpin. These are referred to herein as co-suppression effectors.

A co-suppression effector in the form of a “hairpin-shaped” comprises the spacer nucleotide sequence flanked on each side by an IR sequence capable of annealing to form a double-stranded portion. Where such annealing occurs, the co-suppression effector takes the form of a double-stranded portion and a loop portion, thus resembling a hairpin in shape.

As mentioned above, the spacer loop portion of a co-suppression construct or vector need not necessarily comprise an intron. However, where it does, subsequent transcription and restriction of the double-stranded form thereof results in the creation of RNA molecules which comprise a double-stranded nucleotide sequence, from which the single-stranded loop, which would have been an intron, has been spliced out. The co-suppression effector RNA molecule thus formed is referred to herein as a “perfect” hairpin, inasmuch as the stem-loop structure of the “hairpin” has been altered to yield only the double-stranded stem portion.

In another embodiment, the present invention provides a mixture of nucleic acid co-suppression effector molecules in the form of double-stranded RNA optionally with a single-stranded loop portion, formed by in vitro transcription and/or processing of a co-suppression construct.

Accordingly, another aspect of the present invention contemplates a method for generating a co-suppression library of viral- or eukaryotic-derived nucleic acid molecules in a suitable cell, said method comprising the steps of:—

(i) generating a vector wherein, in vitro, the vector comprises a co-suppression vector having a single-stranded loop portion and a single-stranded replicon portion separated by a double-stranded portion comprising at least one restriction endonuclease site;

(ii) digesting the double-stranded portion of the vector with the at least one restriction endonuclease and admixing therewith a double-stranded genomic DNA or cDNA preparation digested with the same restriction endonuclease or with an enzyme or under conditions providing compatible 3′ and 5′ end portions for ligation into the restricted co-suppression vector and subjecting said admixture to ligation conditions to generate the co-suppression construct comprising double-stranded genomic DNA or cDNA fragments inserted into the double-stranded portion of said vector; and

(iii) introducing the ligated admixture of (ii) into said suitable cell under conditions to permit the generation of a double-stranded replicative form of said co-suppression construct.

Reference herein to a viral-derived nucleic acid molecule includes reference to nucleic acid molecules derived from a virus such as but not limited to a geminivirus or other plant virus, a retrovirus, a human immuno-deficiency virus or a hepatitis virus, inter alia.

In an alternative embodiment, the double-stranded replicative form of the co-suppression construct is first generated in vitro from the ligated admixture. The replicative form is then subsequently introduced into a suitable cell.

Accordingly, in another embodiment, the present invention contemplates a method for generating a library of eukaryotic-derived nucleic acid molecules in a suitable cell, said method comprising the steps of:—

(i) generating a vector wherein, in vitro, the vector comprises a co-suppression vector having a single-stranded loop portion and a single-stranded replicon portion separated by a double-stranded portion comprising at least one restriction endonuclease site;

(ii) digesting the double-stranded portion of the vector with the at least one restriction endonuclease and admixing therewith a double-stranded genomic DNA or cDNA preparation digested with the same restriction endonuclease or with an enzyme or under conditions providing compatible 3′ and 5′ end portions for ligation into the restricted co-suppression vector and subjecting said admixture to ligation conditions to generate the co-suppression construct comprising double-stranded genomic DNA or cDNA fragments inserted into the double-stranded portion of said vector; and

(iii) generating, in vitro, a double-stranded replicative form of the co-suppression construct from the ligated admixture of (ii), prior to introducing the resulting double-stranded replicative form into said suitable cell.

In one preferred embodiment, the vector of step (i) is an expression vector.

A library may be made in vectors that already contain eukaryotic promoters and other regulatory sequences, or a library may be made first in a prokaryotic vector and then the inverted repeats may be re-cloned into an expression vector.

The co-suppression library, therefore, comprises co-suppression constructs, comprising therein eukaryotic DNA in double-stranded form. The library may be in, for example, a prokaryotic microorganism or it may be in an isolated purified form.

When in the latter form, the co-suppression library may then be introduced into a suitable eukaryotic cell or, more generally, a culture of eukaryotic cells or a eukaryotic cell line. The selection of eukaryotic cell is dependent on the trait for which PTGS or TGS is sought. Such traits include loss of enzyme function, alteration in cell surface receptors, change in the colour of a plant, flower or petal, an alteration in the level of resistance to a pathogen, inhibition or promotion of apoptosis, amongst many others.

The present invention contemplates, therefore, a method for identifying a eukaryotic-derived nucleic acid molecule capable of inducing PTGS or TGS in a eukaryotic cell, said method comprising:—

(i) generating a vector wherein, in vitro, the vector comprises a co-suppression vector comprising a single-stranded loop portion and a single-stranded replicon portion separated by a double-stranded portion comprising at least one unique restriction endonuclease site;

(ii) digesting the double-stranded portion of the co-suppression vector with the at least one restriction endonuclease enzyme and admixing eukaryotic-derived DNA having compatible 5′ and 3′ ends for ligation into the 5′ and 3′ ends of the digested co-suppression vector;

(iii) introducing the resulting ligated single-stranded co-suppression construct into a suitable cell to generate a double-stranded form of the co-suppression construct comprising the eukaryotic DNA; and

(iv) isolating the double-stranded co-suppression construct from said suitable cell and introducing same into a eukaryotic cell or eukaryotic cell line and screening for a trait change in said eukaryotic cell wherein the presence of a trait change is indicative of TGS or PTGS.

Generally, but not necessarily, the suitable cell in this embodiment is a prokaryotic microorganism.

With respect to each of the aforementioned embodiments and not intending to limit the present invention in any way, it is proposed that, in vivo, the vector of step (i) may alternatively be in the form of a single-stranded circular molecule.

Introduction of the double-stranded form of the co-suppression construct, comprising the eukaryotic-derived DNA, into a eukaryotic cell or eukaryotic cell line and permitting expression thereof via a promoter operable in the eukaryotic cell and operably linked upstream of the IRs results in an RNA transcript comprising double-stranded RNA with or without a stem loop. It is this which is referred to as a co-suppression effector and which is proposed to induce TGS and/or PTGS via, for example, RNAi.

The present invention further contemplates using a co-suppression construct, identified by the above method, for the production of transformed eukaryotic cells, tissues or group of tissues that may subsequently be regenerated into an organism exhibiting a desired trait change. Accordingly, having screened for a desired trait change, brought about by the action of a co-suppression effector in a cell following introduction into the cell of a double-stranded co-suppression construct, the double-stranded co-suppression construct that caused the desired trait change may be identified and isolated. This double-stranded co-suppression construct may then be employed in the production of stably transformed eukaryotic organisms exhibiting the desired selected trait.

The co-suppression construct or a co-suppression library may also be packaged for sale with instructions for use and/or may be provided in the form of a kit.

Kits made in accordance with the present invention may be used for the production of one or more desired IR DNA constructs, referred to herein as co-suppression constructs.

The present invention further provides cultures of prokaryotic microorganisms comprising the co-suppression constructs or co-suppression library. The present invention further provides eukaryotic cells exhibiting TGS or PTGS of a particular gene.

A further feature of the invention is the use of the co-suppression constructs and/or effectors made in accordance with the method of the invention as actives in a pharmaceutical composition. An isolated co-suppression construct and/or effector of the invention may be used as an active in a pharmaceutical composition. The nucleic acid construct and/or effector may be either DNA or RNA. Preferably, the nucleic acid is RNA. Alternatively, or in addition, an expression vector comprising a nucleic acid co-suppression construct which, when expressed, forms a co-suppression effector may also be used in a pharmaceutical composition.

The pharmaceutical forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of superfactants. The preventions of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization. In the case of sterile powders for the preparation of sterile injectable solutions, suitable methods of preparation include vacuum drying and the freeze-drying technique which yield a powder of active ingredient plus any additionally desired ingredient.

When the active ingredient is suitably protected, it may be orally administered, for example, with an inert diluent or with an assimilable edible carrier, or it may be enclosed in hard or soft shell gelatin capsule, or it may be compressed into tablets, or it may be incorporated directly with the food of the diet or administered via breast milk. For oral therapeutic administration, the active ingredient may be incorporated with excipients and used in the form of ingestible tablets, buccal tablets, troches, capsules, elixirs, suspensions, syrups, wafers and the like. Such compositions and preparations should contain at least 1% by weight of active compound. The percentage of the compositions and preparations may, of course, b varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 200 mg of active compound. Alternative dosage amounts include from about 1 μg to about 1000 mg and from about 10 μg to about 500 mg. These dosages may be per individual or per kg body weight. Administration may be per hour, day, week, month or year.

The tablets, troches, pills, capsules, creams and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, their use in the therapeutic compositions is contemplated. Supplementary active ingredients can also be incorporated into the compositions.

It is particularly advantageous to incorporate the active ingredient as a cream capable of preventing or delaying aging.

The present invention is further described by the following non-limiting Examples.

### EXAMPLE 1

### General Materials and Methods

Molecular biological methods and reagents used herein which are common in the art are described in Sambrook et al. (Molecular Cloning. A Laboratory Manual (Cold Spring Harbor Press, 1989) and Ausubel et al. (Current Protocols in Molecular Biology. Eds, John Wiley & Sons, Inc., 1995-1999) both incorporated herein by reference.

### EXAMPLE 2

### Preparation of Competent Cells

Inoculate 1 L of L-broth with 1/100 volume of fresh overnight culture. Grow cells at 37° C. with vigorous shaking to an A₆₀₀ of 0.5 to 0.8. To harvest, chill the flask on ice for 15 to 30 minutes, and centrifuge at 4000 g for 15 minutes. Cells are kept as close to 0° C. as possible throughout their preparation. Remove as much of the supernatant (medium) as possible. Resuspend pellets in a total of 1 L of ice-cold water. Centrifuige at 4000 g for 15 minutes. Resuspend in ˜20 ml of ice cold 10% v/glycerol. Centrifuge at 4000 g for 15 minutes and resuspend to a final volume of 2 to 3 ml in ice cold 10% glycerol. This suspension may be frozen in aliquots on dry ice, and stored at −70° C.

Gently thaw the cells at room temperature and then immediately place them on ice. Remove sterile cuvettes from their pouches and place them on ice. Place the white chamber slide on ice. In a cold, 1.5 mL polypropylene tube, mix 40 μL of the cell suspension with 1 to 2 μL of DNA. Mix well and let sit on ice ˜0.5 to 1 minute. Set the Gene Pulser apparatus at 25 μF. Set the Pulse Controller at 200 Ω. Set the Gene Pulser apparatus to 2.50 kV when using 0.2 cm cuvettes. Set it to 1.50 to 1.80 kV when using the 0.1 cm cuvettes. Transfer the mixture of cells and DNA to a cold electroporation cuvette, and shake the suspension to the bottom. Place the cuvette in a chilled safety chamber slide. Push the slide into the chamber until the cuvette is seated between the contacts in the base of the chamber. Pulse once at the above settings.

### EXAMPLE 3

### Construction of a Replicative DNA Cloning Vector

Standard molecular biological methods and reagents used for cloning are described in Sambrook et al. (1989, supra) and Ausubel et al. (1994, supra) incorporated herein by reference.

Essentially any vector, capable of being converted to a single-stranded form, can be used as a starting point into which a nucleic acid fragment may be cloned in such a manner as to generate an inverted repeat (IR) of the cloned nucleic acid. For example, a spacer DNA encoding, for example, an intron can be cloned into the multiple cloning site (MCS) of pBluescript (Stratagene, USA). A fragment of the MCS can then be cloned into this plasmid using standard molecular biological methods so that an IR of a region of the MCS flanks the spacer. pBluescript per se provides useful elements: an f1 origin of replication and selectable marker, for example. Details on how to create such a DNA cloning vector are provided below.

The MCS from pCMV-PCR (Stratagene, USA) is another useful sequence for providing several restriction enzyme recognition sites for constructing an IR sequence. Additional useful elements, other than those present in pBluescript, can be included as part of a DNA cloning vector; for example, a spacer nucleic acid, a selectable marker and a eukaryotic and/or prokaryotic promoter, operably linked upstream of an IR. As already mentioned, a spacer nucleic acid may encode an intron. Preferably, the intron is selected to correspond to an organism which is ultimately to express a co-suppression construct as described herein. The spacer may comprise any number of nucleic acids as selected by a skilled person in the art.

One useful spacer nucleic acid comprises a polyphenol oxidase (ppo) intron. The ppo intron was amplified via PCR using a genomic PPO clone as template and primers ppoAcc and ppoBsi, described below. The primers contain regions of sequence complementarity to the PPO gene and have additional sequences which encode a restriction enzyme recognition site for Acc65I (GGTACC) [SEQ ID NO:I] or BsiWI (CGTACG) [SEQ ID NO:2]. PCR products were digested with Acc65I and BsiWI restriction enzymes, separated by gel electrophoresis on an agarose gel and a 640 bp fragment was purified by standard molecular biological techniques. The resulting four-base 5′-overhangs produced by both enzymes are compatible with Acc65I-derived overhangs.

| | | |
|---|---|---|
| Ppo | 5′-GTCAGGTACCGACAGGTAATCGCGT-3′ | [SEQ ID |
| Acc | | NO:3] |
| Ppo | 5′-GTCTTCGTACGGGATATCACCTGTCAAAATC-3′ | [SEQ ID |
| Bsi | | NO:4] |

pBluescript SK⁻ DNA was digested with Acc65I and the 5′-termini were dephosphorylated using shrimp alkaline phosphatase. The dephosphorylated pBluescript SK⁻ DNA was ligated with the 640 bp Acc65I/BsiWI fragment of ppo gene and used to transform E. coli. Recombinants were selected, plasmid DNA was isolated, digested with BssHH/IAcc65I and the 780 bp BssHII/Acc65I fragment (A) was purified.

pBluescript KS⁻ was digested with BssHII and Acc65I and the respective 140 bp BssHII/Acc65I (B) and 2.8 kb BssHII fragments were isolated. The 2.8 kb fragment was dephosphorylated (C) using shrimp alkaline phosphatase. Equimolar amounts of (A), (B) and (C) were ligated and used to transform E. coli. A recombinant comprising a desired DNA cloning vector, designated pIR, was selected.

Details in relation to pCMV-PCR, pBluescript and other useful vectors are commercially available and information in relation to these vectors may be provided by the supplying company. Such commercial product information is herein incorporated by reference.

DNA cloning vectors containing IRs and useful sites can be created in other ways. The following describes another method of creating such a vector, starting with pCMV-PCR.

pCMV-PCR plasmid DNA was digested with MluI/DraIII and the 5′-termini were dephosphorylated by treatment with shrimp alkaline phosphatase. The multiple cloning site of pCMV-PCR was amplified via PCR, using the oligonucleotide primers MCS-Sac-Dra and MCS-Kpn-MluI. The amplified MCS fragment was purified with a QIAquick PCR purification column and digested with DraIII and MluI. The 150 bp DraIII/MluI MCS fragments were gel purified and ligated to the MluI/DraIII-digested pCMV-PCR. The ligation mix is used to transform E. coli cells. Recombinant clones, which contain an inverted repeat of the multiple cloning site, separated by a spacer region of 440 bp, are selected. The resultant vector is designated pCMV-IR. The BbvCI site within pCMV-IR (generated from the MCS-Sac-Dra PCR primer) allows production of single-stranded IR cloning vector DNA via nicking with N.BbvCIA or N.BbvCIB, followed by Exonuclease III digestion, similar to the process described in Example 6.

Oligonucleotide Sequences:

| | | |
|---|---|---|
| MCS-Sac-Dra | 5′-GGGAACACGTAGTGCTGAGGAGCTCCACCGCGGTGGC-3′ | [SEQ ID NO:7] |
| MCS-Kpn-Mlu | 5′-ACTTAACGCGTACCGGGCCCCCCCTC-3′ | [SEQ ID NO:8] |

The inverted repeats within the final vector contain two SapI sites. Digestion of annealed single-stranded form of pCMV-IR with SapI generates a vector stem/loop, a smaller spacer stem loop and a very small linear double-stranded fragment. The SapI-digested vector stem loop terminus contains a 5′-CTT-overhang, the spacer/loop terminus contains a 5′-CGG-overhang and the released double-stranded linear fragment contains 5′-AAG- and 5′-CCG-overhangs. The linear fragment can conveniently be removed from the restriction digestion by, for example QIAquick PCR column purification.

Ligation of added double-stranded DNA (eg. cDNA or genomic DNA) with compatible ends (i.e.: containing a 5′-AAG- and a 5′-CCG-overhang) to the pCMV-IR stem/loop fragments, followed by conversion to double-stranded form results in the creation of inverted repeat constructs of the added DNA fragments. The vector stem/loops cannot ligate to each other and the spacer stem/loops cannot ligate to each other, and the added DNA fragments cannot ligate to each other. Double-stranded DNA fragments with compatible ends for cloning into pCMV-IR can be made by addition primer sequences via PCR, ligation of adaptor sequences or by first cloning blunt-end fragments into EcoRV-cut pCMV-PCR, followed by digestion with SapI and purification of resultant fragments.

Further modifications to the pCMV-IR vector can be made to reduce background resulting from undigested pCMV-IR in the ligations of SapI-digested single-stranded vector to compatible double-stranded DNA fragments. Cloning of a ‘killer’ gene such as ccdB between the SapI sites of the inverted repeats creates an inverted repeat of the ccdB gene, which is removed from the vector by SapI digestion. An inverted repeat cloning vector containing the ccdB gene can be propagated in DB3.1 E. coli cells and then converted into single-stranded inverted repeat cloning vector. However, any plasmids containing the ccdB gene are not able to propagate in wild-type E. coli strains which lack an F′ episome such as DH5-alpha or TOP10. This provides an efficient negative selection method for eliminating non-recombinant clones from an inverted repeat cloning experiment.

A DNA cloning vector may comprise the following:

(i) Restriction endonuclease recognition sites, T-overhang, LIC, and other useful sites that may be selected by a skilled person in the art.

(ii) An IR which flanks both sides of a ‘spacer’ region of DNA. Preferably the spacer is longer than 300 bp. More preferably the spacer encodes an intron.

(iii) The capability for single-stranded rescue of the vector and insert, via filamentous bacteriophage DNA replication control elements (e.g. the intergenic region from f1 phage or a vector constructed from a filamentous phage such that rescue is not needed).

(iv) A fully functional replicon so that replication of the vector may occur in a permissive host organism.

(v) A site for a “nicking” restriction enzyme, such as N.Bpu1OI, N.BbvCIA or N.BbvCIB.

An example of cloning steps to produce a DNA cloning vector is illustrated diagrammatically in FIG. 7. Vectors and DNA fragments represented are double-stranded.

### EXAMPLE 4

### Removing or Changing a Spacer Region (Intron) Nucleic Acid

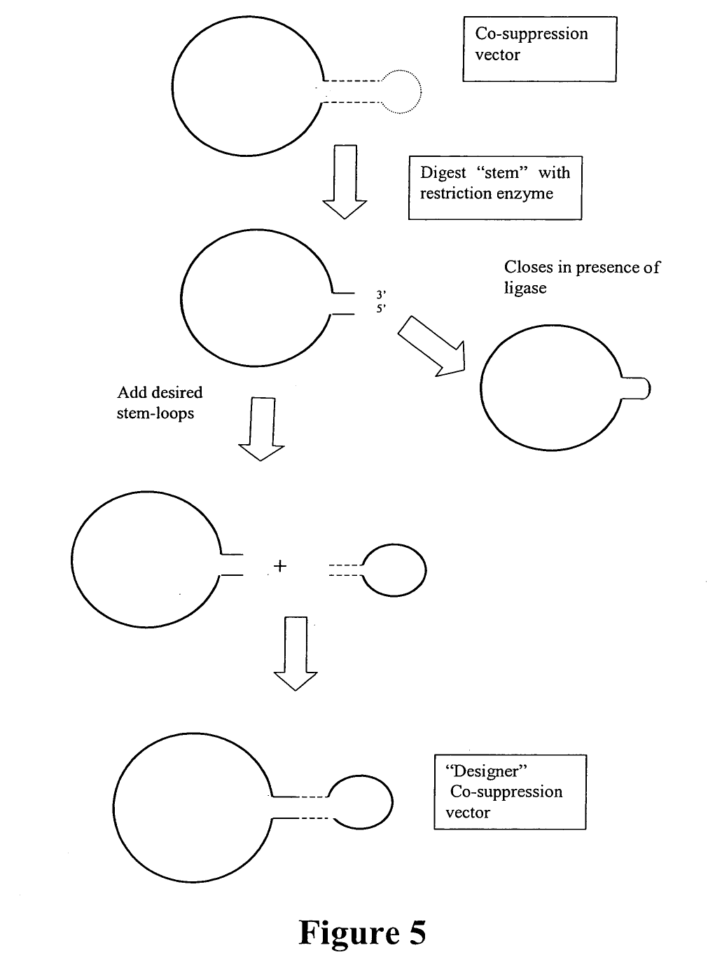

As described in Example 3, above, spacer nucleic acid may be added during the production of DNA cloning vector. A spacer nucleic acid may allow replication in bacteria. Intron-encoding spacers appear to provide greater PTGS efficiency. However, a spacer is not necessarily required if the IR is small (e.g. less than 75 nucleotides), as shown in FIG. 5. Small inverted repeats are generally more stable in sbcC mutant strains of E. coli, such as Sure™ cells.

Removing from or changing a spacer in a DNA cloning vector can be readily achieved. The spacer can be replaced by digestion of the vector with a restriction enzyme that cleaves either side of the spacer, within the IRs. A new spacer DNA fragment with compatible ends can then be added by ligation.

Removal of the spacer altogether can be achieved by self-ligation of the DNA cloning vector after the spacer is removed by restriction digestion.

A spacer can also be replaced in the single-stranded co-suppression vector—see Example 5, below—by ligation of a spacer stem-loop with a compatible replicon (plasmid) stem-loop fragment.

### EXAMPLE 5

### Generation of a Co-Suppression Vector via Single-Stranded Helper Phage

(a) Growth of Bacteria

E. coli strain used was XL-1 Blue XRF′ (A(mcrA)183 Δ (mcrCB-hsdSMR-mrr)173endA1 supE44 thi-1 recA1 gyrA96 relA1 lac[F′ proAB lacl^{q} Z ΔM15 Tn10 (Tet^{r})]). Bacterial strains were grown in under standard conditions. Helper phage VCSM13 (Stratagene) was used for rescue of single-stranded phagemids.

(b) Single-Stranded Phagemid Rescue and Purification

Inoculate a single colony into 5 mL of 2YT containing 50 mg/mL ampicillin and VCSM13 helper phage at 10⁷-10⁸ pfu/mL (multiplicity of infection 10). Grow the culture at 37° C. with vigorous aeration for 16 hours, or until growth has reached saturation. If using VCSM13, after 1-2 hours, add kanamycin to 70 μg/mL to select for infected cells. Centrifuge 1.5 mL of the cell culture for 5 minutes n a microcentrifuge. Remove 1 mL of the supernatant to a fresh tube, then add 150 μL of a solution containing 20% PEG8000 and 2.5 M NaCl. Allow phage particles to precipitate on ice for 15 minutes. Centrifuge for 5 minutes in a microcentrifuge. Remove supernatant and resuspend the pellet in 400 μL of 0.3 M NaOAc (pH 6.0) and 1 mM EDTA by vortexing vigorously. Extract with 1 volume of phenol-chloroform and centrifuge for 1-2 minutes to separate phases. Transfer the aqueous phase to a fresh tube and add 1 mL of ethanol. Centrifuge for 5 minutes. Remove ethanol and dry the pellet comprising the single-stranded form of the DNA cloning vector. Dissolve the pellet in 25 μL of TE buffer.

(c) Digestion of ‘Single-Stranded’ Co-Suppression Vector

To ensure the production of a partially double-stranded stem portion within the ‘single-stranded’ co-suppression vector DNA, phagemid DNA was heated to 90° C. for 3 minutes and allowed to cool slowly to room temperature (30-60 minutes). An aliquot of this sample was digested with 2 units of restriction enzyme per microgram of phagemid DNA in a volume of 20 μL for 1 hour.

### EXAMPLE 6

### Generation of a Co-Suppression Vector, In Vitro

To generate a co-suppression vector in vitro, the following components were added to the reaction tube:

| | | |
|---|---|---|
| plasmid containing Bpu10I site (10 μg) | 10-184 | μl |
| 10X R+ reaction buffer | 20 | μl |
| N.Bpu10I (5 u/μl) | 2 | μl |
| Deionized water up to | 200 | μl |

The tube was vortexed and spun in a microcentrifuge for 3-5 sec and then incubated at 37° C. for 1 hr. Following incubation, 12 volumes of phenol (100 μl) and 12 volume of chloroform/isoamyl alcohol (24:1) (100 μl) were added, and the mixture was, vortexed for 10 sec and then centrifuged at maximum speed for 5 min.

The upper aqueous phase was transferred to a fresh tube and 1 volume (200 μl) of chloroform/isoamyl alcohol (24:1) was added. The mixture was vortexed and centrifuged for 5 minutes. Following two further extractions with chloroform/isoamyl alcohol, the upper aqueous phase was transferred to a fresh tube. A 1/10 volume of 3M sodium acetate and 2.5 volumes of ice-cold ethanol were added and the mixture incubated at −20° C. for 1 hr. Following incubation, the mixture was centrifuged at maximum speed for 10 minutes.

The supernatant was poured off and the DNA pellet carefully washed with 200 μl 75% ice-cold ethanol, dried and dissolved in 25 l of deionized water. Exonuclease HIT treatment was achieved by adding to the 25 μl nicked DNA the following components: 12.5 μl 10×ExoIII reaction buffer, 3 μl Exonuclease III (200 U/μl) and 5 μl deionized water. This mixture was incubated for 15 min at 37° C. and the reaction stopped by heating stopped by heating at 70° C. for 10 min.

Finally, the resulting DNA was cleaned by purification using a QIAquick PCR kit (Qiagen) and eluted in 30 μl EB or deionized water.

### EXAMPLE 7

### Ligation to Double-Stranded DNA Fragments to Form a co-Suppression Construct

Double-stranded DNA fragments with compatible ends are added to the purified restriction enzyme digested products of the ‘single-stranded’ co-suppression vector and ligated using standard conditions (Sambrook et al., 1989, supra).

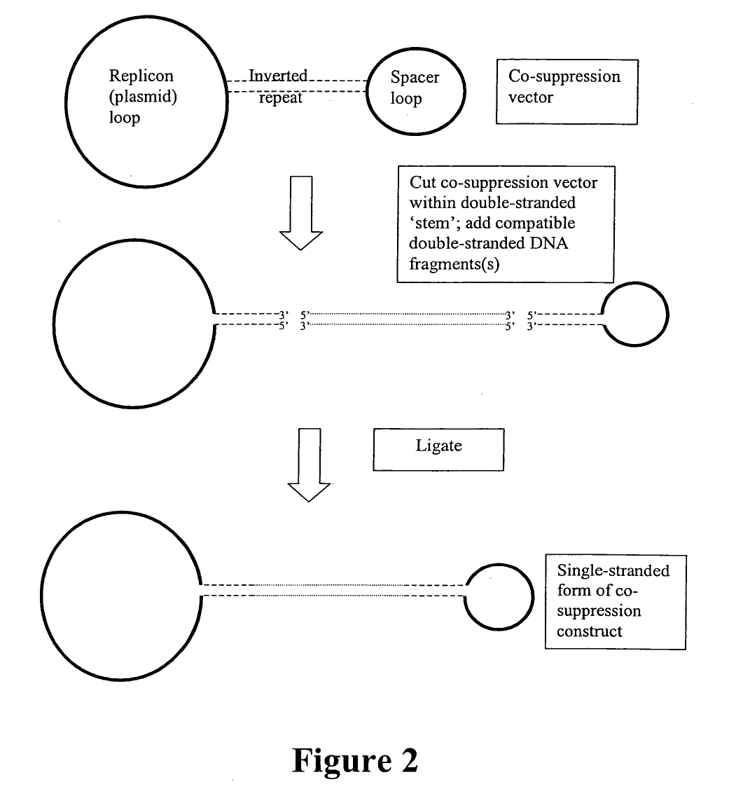

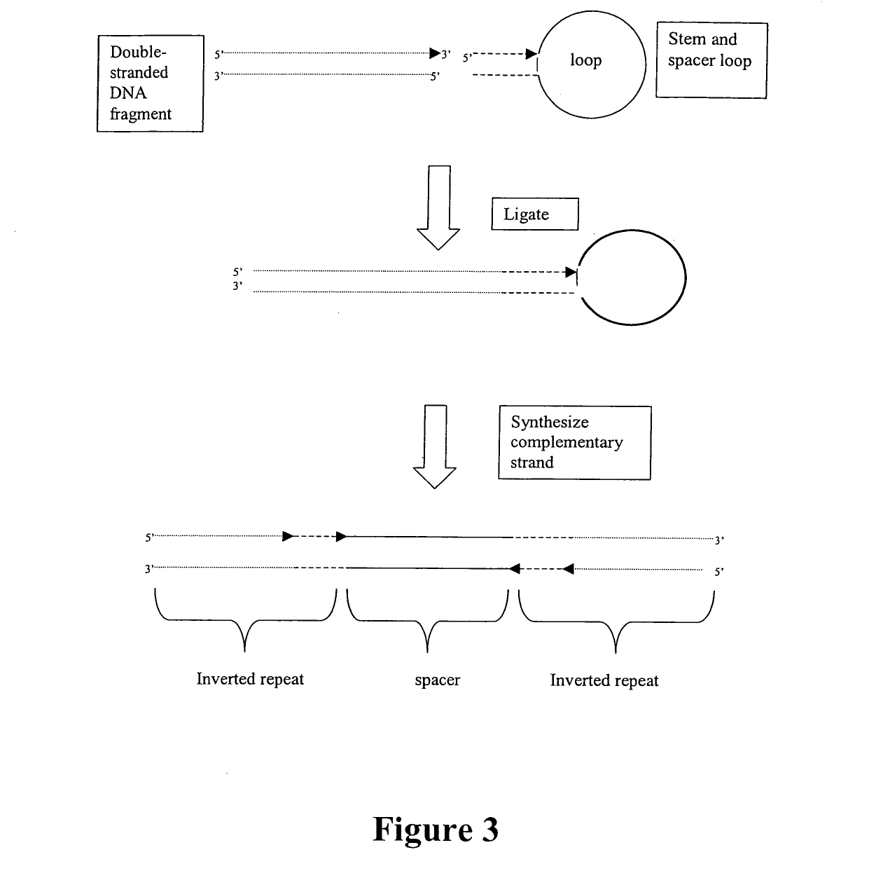

Co-suppression constructs can be created by cloning double-stranded DNA fragments into the IR region of the co-suppression vector. Conversion of the single-stranded recombinant DNA co-suppression construct into the ‘double-stranded’ form generates an IR of the cloned DNA. The cloned DNA fragment may be inserted into the IR region or may replace part of the IR region. These DNA fragments may be cloned in a variety of ways, some of which are outlined below and illustrated in FIGS. 2-4.

(a) Single Restriction Enzyme Cutter

A co-suppression vector is digested with a restriction enzyme that cleaves within the double-stranded IR region to produce plasmid (replicon) and spacer loops. DNA fragments comprising compatible ends are ligated to the loops to generate a new double-stranded IR region.

One method to increase efficiency of recombinants is to use a DNA cloning vector which comprises a restriction enzyme recognition site which occurs infrequently in genomic sequences (e.g. SrfI). Blunt-ended DNA fragments can then be ligated with SrfI-digested DNA cloning vector in the presence of SrfI enzyme. SrfI sites regenerated by ligation of the stem-and-loops to each other are cut by SrfI, whereas recombinants arising from the ligation of double-stranded DNA fragments to the stem-and-loops destroy the SrfI site. SrfI-containing fragments are not clonable using this method.

(b) Directional Cloning with Two Restriction Enzymes

A co-suppression vector is digested with two restriction enzymes that cleave the double-stranded IR region to produce a plasmid (replicon) stem-loop portion and a spacer stem-loop portion and double-stranded DNA fragment(s) arising from the cleavage. DNA fragments containing compatible ends are ligated to the loops to generate a new double-stranded region. Using two different restriction enzyme sites overcomes the problem of the unwanted re-ligation of the plasmid and spacer loops to each other.

(c) TA-Cloning Method

A co-suppressionvector is digested with a restriction enzyme that cleaves within the IR region to produce plasmid (replicon) and spacer stem-loop portions. Single T 3′-overhangs are added by one of the following methods:

1. digestion of plasmid with e.g. XcmI sites engineered to produce single T 3′-overhangs at the stem termini; or

2. incubation with DNA polymerase in the presence of dTTP (Marchuk et al., Nucleic Acids Research 10: 1154, 1991). The plasmid DNA molecules are then ligated with DNA fragments containing single A 3′-overhangs. A-overhangs can be conveniently generated during or after PCR amplification of DNA fragments.

(d) LIC Adaptors

A co-suppression vector is digested with a restriction enzyme (e.g. SapI) that cleaves within the IR region to produce plasmid and spacer stem-loop portions. The digested DNA is treated with a DNA polymerase in the presence of limiting dNTPs (e.g. only dTTP) to generate single-stranded overhangs. Double-stranded DNA containing single-stranded overhangs compatible with the prepared vector stem-loop portions is annealed with the vector portions to form the co-suppression construct.

Co-suppression constructs generated by any of these means may be used to transform bacteria such as, for example E. coli. Therein the constructs are converted to the double-stranded form, producing a library of co-suppression constructs (ii) (see Example 8, below).

### EXAMPLE 8

### Generation of Stem and Loop Structures by PCR

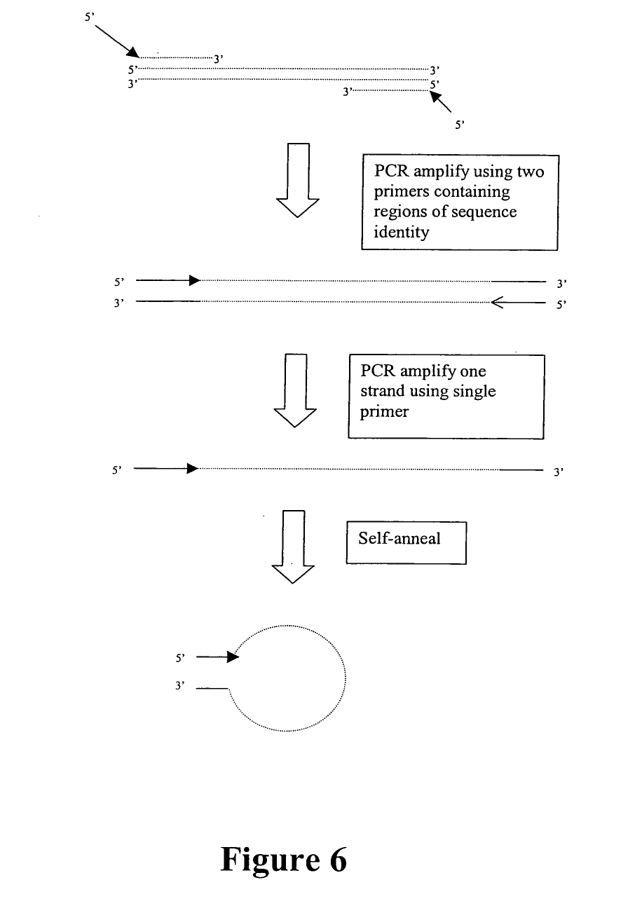

Stem-and-loop structures can also be generated via PCR amplification (see FIG. 6). As an example, the following oligonucleotides were synthesized to amplify a pineapple polyphenol oxidasae (PPO) intron:

| | |
|---|---|
| PPO-Srf-F: | |
| 5′-CCCGTGCTCCGACAGgtaatcgcgttag-3′ | [SEQ ID NO:5] |
| PPO-Srf-R: | |
| 5′-CCCGTGCTCCATCACctgtcagggtcgcaat-3′ | [SEQ ID NO:6] |

The underlined sequences are not present in the PPO gene sequence but were added to create terminal IRs in the PCR amplification products.

The pineapple PPO intron as amplified via PCR using the oligonucleotides PPO-Srf-F and PPO-Srf-R as primers. Further amplification in the presence of only PPO-Srf-F results in the amplification of only one DNA strand, leading to the production of predominantly single-stranded DNA flanked by 10-nucleotide IRs. Incorporation of the underlined sequence into the amplification products can form stem and loop structures. These can be ligated to blunt-ended double-stranded DNA fragments and/or to replicon-stem portions to form a co-suppression construct.

In an alternative approach, a single-stranded spacer-loop with self-complementary ends (i.e. the double-stranded stem portion) may be ligated to a single-stranded cDNA polynucleotide and used to prime second strand synthesis. A similar approach may be taken using single-stranded genomic DNA or PCR fragments. Once the double-stranded stem portion has been generated, these may again be ligated to double-stranded DNA fragments and/or to replicon-stem portions to form a co-suppression construct.

### EXAMPLE 9

### Hybrid Co-Suppression Vectors

Hybrid co-suppression vectors are generated comprising replicons (plasmids) and stem-loops from different co-suppression vectors. In one embodiment, a co-suppression vector is subjected to rolling-circle replication in vitro to generate a concatamer comprising multiple repeats of a strand of the co-suppression vector. The concatamer is subjected to annealing conditions to provide a nucleic acid complex having multiple stem-loops portions. This complex is used as a source of stem-loops for ligation to a suitably-digested co-suppression vector.

In an alternative embodiment, the starting material comprises single dsDNA cloning vectors. Stem-loops may also be generated in vitro via, for example, the use of PCR. In a preferred embodiment, replicon stem-loops and spacer stem-loops each contain a different selectable marker. For example, ligation of a replicon stem-loop containing an ampicillin resistance gene and a spacer stem-loop containing a kanamycin resistance gene generates a recombinant vector which is both ampicillin resistant and kanamycin resistant. When the two stem-loops are generated, derived from separate plasmids, and ligated in the presence of ds fragments, recombinants may easily be selected, eliminating the background of non-recombinants.

### EXAMPLE 10

### Conversion of Recombinant Co-Suppression Construct (i) to Double-Stranded Form (ii)

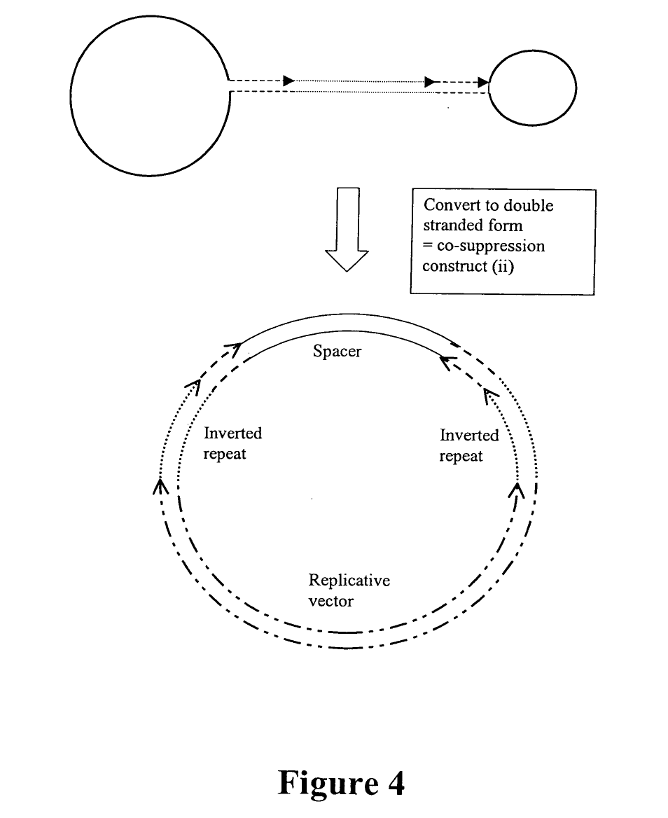

The ligation mix comprising co-suppression constructs (i) is used to transform E. coli. The recombinant co-suppression construct replicates in E. coli and is converted to the double-stranded form (ii) and, in the process, generates an IR of the cloned DNA fragment flanking the spacer region (refer to FIG. 4).

A single stranded co-suppression construct (i) can also be converted to a predominantly double-stranded form (ii) in vitro, prior to bacterial transformation. Conversion to double-stranded form can be achieved by annealing a complementary oligonucleotide primer and extending with a DNA polymerase such as Taq.

A PCR reaction mix consisted of:

10 μl of annealed single-stranded co-suppression vector,

5 μl of 10×PCR reaction buffer,

1 U Taq DNA polymerase,

0.2 μM primer,

250 μM each dATP, dCTP, dGTP, dTTP,

deionized water to make up final reaction volume to 50 μl.

The reactions were cycled as follows: 95° C. for 30 sec, 50° C. for 30 sec, 72° C. for 7 min. Reactions were cleaned up using QIAgen mini-elute columns: purified DNA was eluted in 10 μl EB buffer and used for E. coli transformation. Similar PCR conditions were also used to convert single-stranded ligated DNA to double-stranded form before transformation of E. coli.

### EXAMPLE 11

### Creation of a Co-Suppression Library via Shot-Gun Cloning

A library of DNA fragments (cDNA or genomic) corresponding to a single gene or genetic cluster, may be generated by isolating the DNA, restricting the DNA to generate a range of differently-sized fragments, size-fractionating the fragments and selecting a particular size range thereof for cloning into a suitably-digested co-suppression vector. The double-stranded DNA fragments can be generated in a variety of ways familiar to those skilled in the art. These methods include restriction digestion, sonication, partial cleavage with DNAse I, PCR amplification of template DNA, synthesis of cDNA from RNA. Double-stranded DNA fragments with termini compatible with cloning into a co-suppression vector can be generated in many ways, including restriction digestion, adaptor ligation, PCR amplification and end-repair of DNA fragments.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ausubel et al., Current Protocols in Molecular Biology, Eds. John Wiley & Sons, Inc., 1995-1999

Bingham, Cell 90: 385-387, 1997;

Caplen et al., Gene 252: 95-105, 2000;

Cogoni and Mancino, Proc. Natl. Acad. Sci. USA 94: 10233-10238, 1997;

Cogoni et al., EMBO J. 15: 3153-3163, 1996;

Elbashir et al., Nature 411: 494-498, 2001;

Fire et al., Nature 391: 806-811, 1998;

Forthler and Belote, Genesis 264: 240-244, 2000;

Lindbo et al., The Plant Cell 5: 1749-1759, 1993;

Marchuk et al., Nucleic Acids Research 10: 1154, 1991;

Napoli et al., The Plant Cell 4: 279-289, 1990;

Sambrook et al., Molecular Cloning: A Laboratory Manual, Cold Spring Harbor Press, 1989;

Schoppmeier and Daman, Development Genes and Evolution 211: 76-82, 1001;

Singh et al., Biochemical Society Transactions 28: 925-927, 2000;

Smith et al., Nature 407: 319-320, 2000;

Timmons and Fire, Nature 395:854, 1998;

Timmons et al., Gene 263: 103-112, 2001;

van der Krol et al, The Plant Cell 4: 291-299, 1990;

Waterhouse et al., Proc. Natl. Acad. Sci. USA 95: 13959-13964, 1999;

1. A method for generating a library of viral- or eukaryotic-derived nucleic acid molecules in a suitable cell, said method comprising the steps of:—
(i) converting a double-stranded replicative circular DNA cloning vector, comprising an inverted repeat (IR) sequence, into a single-stranded form;
(ii) treating said single-stranded form such that self-complementary sequences derived from said IR sequence anneal to form a region of double-stranded nucleic acid;
(iii) cleaving said double-stranded region formed in step (ii) by one or more restriction enzymes to form a vector stem-loop portion and a spacer stem-loop portion;
(iv) ligating said stem-loop portions of step (iii) with double-stranded DNA fragments containing termini compatible with said vector and spacer stem-loops to form recombinant nucleic acid molecules; and
(v) converting recombinant nucleic acid molecules of step (iv) into a double-stranded circular form.

2. A method of claim 1, wherein the double-stranded region formed in step (ii) contains at least one restriction enzyme recognition site.

3. A method of claim 1, wherein said nucleic acid molecules of step (iv) are converted into a double-stranded circular form in vitro.

4. A method of claim 1, wherein said nucleic acid molecules of step (iv) are converted into a double-stranded circular form by transformation of a host cell capable of carrying out said conversion as part of the replicative process.

5. A method of any one of claims 1 to 4, wherein said suitable cell is a prokaryotic microorganism.

6. The method of any one of claims 1 to 4, wherein said suitable cell is a eukaryotic cell.

7. A method for generating a library of viral- or eukaryotic-derived nucleic acid molecules in a suitable cell, said method comprising the steps of:—
(i) generating a vector wherein, in vitro, the vector comprises a single-stranded replicon portion and a single-stranded loop portion separated by double-stranded stem portion comprising at least one restriction endonuclease site;
(ii) digesting said partially single-stranded vector with at least one restriction endonuclease and admixing therewith double-stranded genomic DNA or cDNA derived from a eukaryotic cell and digested with the same restriction endonuclease or other enzyme or under conditions providing compatible 3′ and 5′ end portions for ligation into the restricted partially single-stranded vector and subjecting said admixture to ligation conditions to generate the partially single-stranded vector comprising double-stranded genomic DNA or cDNA fragments inserted into the double-stranded portion of said vector; and
(iii) introducing the ligated admixture of (ii) into said suitable cell under conditions to permit the generation of a double-stranded replicative form of said partially single-stranded vector comprising double-stranded genomic DNA or cDNA fragments.

8. The method of claim 7, wherein the double-stranded replicative form of step (iii) is first generated in vitro from the ligated admixture prior to introducing said double-stranded form into said cell.

9. The method of claim 7 or claim 8, wherein said suitable cell is a prokaryotic microorganism.

10. The method of claim 7 or claim 8, wherein said suitable cell is a eukaryotic cell.

11. The method of claim 7 or claim 8, wherein said double-stranded stem portion arises from self-annealing of complementary sequences derived from inverted repeat sequences in a DNA cloning vector.

12. The method of claim 7 or claim 8, wherein the generation of the vector of step (i) is initiated by first obtaining a double-stranded DNA cloning vector having a multiple cloning site such that, upon digestion, said cloning vector is cleaved only within the multiple cloning site.

13. The method of claim 12, wherein a spacer nucleic acid molecule is cloned within the multiple cloning site of the DNA cloning vector.

14. The method of claim 13, wherein the spacer comprises an intron.

15. The method of any one of claims 12, 13 or 14, wherein the DNA cloning vector comprises restriction sites adjacent both sides of said spacer.

16. The method of claim 15, wherein the restriction sites facilitate directional cloning of digested DNA fragments.

17. The method of any one of claims 12 to 15, wherein said DNA cloning vector comprises two homologous nucleotide sequences flanking said spacer such that, when in single-stranded form, said spacer permits said homologous sequences to anneal together to create a partially double-stranded molecule.

18. The method of any one of claims 12 to 16, wherein said DNA cloning vector is capable of generating single-stranded replicative intermediates in the presence of a helper phage.

19. The method of claim 18, wherein said replicative intermediate is generated by nicking one strand of said double-stranded DNA cloning vector and digesting said nicked strand with an exonuclease.

20. The method of any one of claims 7 to 19, wherein said suitable cell supports formation of a single-stranded replicative form via the use of a helper phage.

21. The method of claim, 20, wherein said suitable cell is a prokaryotic microorganism.

22. The method of claim 7 or claim 8, wherein, in vivo, the vector of step (i) is in the form of a single-stranded circular molecule.

23. A co-suppression construct comprising two single-stranded DNA loop portions separated by a double-stranded portion wherein the double-stranded portion comprises one or more restriction endonuclease sites into which has been introduced a double-stranded DNA fragment derived from a eukaryotic cell.

24. The co-suppression construct of claim 23, wherein the eukaryotic cell is derived from a species from the list consisting of plants, invertebrate animals such as insects and nematodes, and vertebrate animals such as mice, livestock and humans.

25. The co-suppression construct of claim 23 or claim 24, wherein said construct is converted into a double-stranded form in a suitable cell.

26. The co-suppression construct of claim 25, wherein said suitable cell is a prokaryotic microorganism.

27. The co-suppression construct of claim 25, wherein said suitable cell is a eukaryotic cell.

28. A method for generating a co-suppression library of viral- or eukaryotic-derived nucleic acid molecules in a suitable cell, said method comprising the steps of:—
(i) generating a vector wherein, in vitro, the vector comprises a co-suppression vector having a single-stranded loop portion and a single-stranded replicon portion separated by a double-stranded portion comprising at least one restriction endonuclease site;
(ii) digesting the double-stranded portion of the vector with the at least one restriction endonuclease and admixing therewith a double-stranded genomic DNA or cDNA preparation digested with the same restriction endonuclease or with an enzyme or under conditions providing compatible 3′ and 5′ end portions for ligation into the restricted co-suppression vector and subjecting said admixture to ligation conditions to generate the co-suppression construct comprising double-stranded genomic DNA or cDNA fragments inserted into the double-stranded portion of said vector; and
(iii) introducing the ligated admixture of (ii) into a suitable cell under conditions to permit the generation of a double-stranded replicative form of said co-suppression construct.

29. The method of claim 28, wherein the double-stranded replicative form of step (iii) is first generated in vitro from the ligated admixture prior to introducing said double-stranded form into said suitable cell.

30. The method of claim 28 or claim 29, wherein said suitable cell is a prokaryotic microorganism.

31. A co-suppression library comprising co-suppression constructs of claim 28 or 29, wherein said co-suppression constructs comprise therein eukaryotic-derived DNA in double-stranded form.

32. The co-suppression library of claim 31 in a prokaryotic microorganism.

33. The co-suppression library of claim 31 in isolated purified form.

34. The isolated co-suppression library of claim 33, comprised in eukaryotic cells or a culture of cells or a cell line.

35. A nucleic acid molecule isolated from the co-suppression library of claim 33 or claim 34.

36. A method for identifying a eukaryotic-derived nucleic acid molecule capable of inducing PTGS or TGS in a eukaryotic cell, said method comprising:—
(i) generating a vector wherein, in vitro, the vector comprises a co-suppression vector comprising a single-stranded loop portion and a single-stranded replicon portion separated by a double-stranded portion comprising at least one unique restriction endonuclease site;
(ii) digesting the double-stranded portion of the co-suppression vector with the at least one restriction endonuclease enzyme and admixing eukaryotic-derived DNA having compatible 5′ and 3′ ends for ligation into the 5′ and 3′ ends of the digested co-suppression vector;
(iii) introducing the resulting ligated single-stranded co-suppression construct into a suitable cell to generate a double-stranded form of the co-suppression construct comprising the eukaryotic DNA; and
(iv) isolating the double-stranded co-suppression construct from the suitable cell and introducing same into a eukaryotic cell or eukaryotic cell line and screening for a trait change in said eukaryotic cell wherein the presence of a trait change is indicative of TGS or PTGS.

37. A method of claim 36, wherein the vector of step (i) is an expression vector.

38. The method of claim 37, wherein, in vivo, the vector of step (i) is in the form of a single-stranded circular molecule.

39. The method of claim 36 or claim 37, wherein said suitable cell is a prokaryotic microorganism.

40. The method of claim 36 or claim 37, wherein said suitable cell is a eukaryotic cell.

41. Use of a co-suppression construct identified by the method of claim 36 in the production of transformed eukaryotic cells, tissues or group of tissues that may subsequently be regenerated into an organism exhibiting a desired trait change.

42. A co-suppression library in the form of a kit packaged for sale and with instructions for use.

43. The kit of claim 42, wherein said kit is used for the production of inverted repeat DNA constructs.


SEQUENCE LISTING (8 sequences)

SEQ ID NO: 1 - DNA, 6 bp, artificial sequence
  ACC65I primer
ggtacc

SEQ ID NO: 2 - DNA, 6 bp, artificial sequence
  BsiWi primer
cgtacg

SEQ ID NO: 3 - DNA, 25 bp, artificial sequence
  PpoAc primer
gtcaggtaccgacaggtaatcgcgt

SEQ ID NO: 4 - DNA, 31 bp, artificial sequence
  PpoBsi primer
gtcttcgtacgggatatcacctgtcaaaatc

SEQ ID NO: 5 - DNA, 28 bp, artificial sequence
  PPO-Srf-F primer
cccgtgctccgacaggtaatcgcgttag

SEQ ID NO: 6 - DNA, 31 bp, artificial sequence
  PPO-Srf-R primer
cccgtgctccatcacctgtcagggtcgcaat

SEQ ID NO: 7 - DNA, 37 bp, artificial sequence
  oligonucleotide
gggaacacgtagtgctgaggagctccaccgcggtggc

SEQ ID NO: 8 - DNA, 25 bp, artificial sequence
  oligonucleotide
cttaacgcgtaccgggccccccctc